(19) United States
(12) Patent Application Publication
(10) Pub. No.: US 2021/0235987 A1
(43) Pub. Date: Aug. 5, 2021

(54) OCULAR BIOMETRY SYSTEMS AND METHODS

(71) Applicant: Auckland UniServices Limited, Auckland (NZ)

(72) Inventor: Seyed Ehsan Vaghefi Rezaei, Auckland (NZ)

(21) Appl. No.: 17/048,919

(22) PCT Filed: Apr. 18, 2019
(86) PCT No.: PCT/NZ2019/050039

§ 371 Date: Oct. 19, 2020

(30) Foreign Application Priority Data
Apr. 20, 2018 (NZ) 741816

(51) Int. Cl. A61B 3/14; A61B 3/00; A61B 3/12; A61B 3/117; A61B 3/107

(52) U.S. Cl. CPC A61B 3/14; A61B 3/0008; A61B 3/107; A61B 3/1173; A61B 3/12

(57) ABSTRACT

Parameters of an eye are measured by capturing images of the eye when at least one light source is shone into the eye and analyzing the captured images. An ocular biometry system includes a light source configured to generate a light beam, cameras configured to capture images of the eye when the light beam passes through the eye, and processors configured to identify features in the captured images. The features represent the light beam passing from one part of the eye to another part of the eye. One or more parameters of the eye are determined from the identified features. The light beam can be adjusted to be incident on the eye in a number of positions and multiple light beams can be used.

### FIELD OF INVENTION

The invention generally relates to the field of ocular biometry. More particularly, the invention relates to systems and methods for measuring eye parameters by capturing images of an eye when a light source is shone into the eye.

### BACKGROUND TO THE INVENTION

Refractive errors in the eye occur when the eye is unable to adequately focus an image on the retina. The result of refractive errors may be blurred images. Types of refractive error include myopia (near-sightedness), hyperopia (far-sightedness), astigmatism and presbyopia. If refractive errors are left uncorrected a person's vision can continue to deteriorate, and may ultimately lead to blindness.

Estimates suggest that uncorrected refractive error is responsible for roughly half of global vision impairment and one of the leading causes of blindness. Myopia alone is estimated to affect 1.5 billion people globally and is a particular problem in Asian countries where it has been estimated to affect approximately 87% of the young adult population, compared to just 35% in the UK. Projections show that the number of people affected by myopia, and other refractive errors, will continue to increase if the issue is not addressed.

Identifying refractive errors and monitoring their progression is critical to diagnosis, prevention and treatment. Robust clinical trials demonstrate that many issues with vision can be prevented if detected early and timely control interventions are instigated. Ideally, ongoing monitoring should be conducted on a regular basis, for example every 2-6 months. However, the time and/or cost of assessments can make regular monitoring difficult. Conventional ‘low-tech’ eye tests (e.g. involving trial and error testing of different lenses or reading letters on a board) by an optometrist or ophthalmologist can take up to an hour. Optical biometry equipment based on interferometry can perform assessments very quickly but such equipment is very expensive.

Also, some existing devices operate on the basis only of detecting light that has passed through a central portion of the eye. Such devices may not detect defects with non-central portions of the eye, for example some astigmatisms.

High levels of myopia have long been associated with the onset of staphyloma in children, and non-reversible blindness. In order to detect staphyloma in children, an accurate imaging of the 3D geometry of the posterior eye is needed, for which optical coherence tomography (OCT) is used. OCT devices are expensive and often result in a lower rate of detection of staphyloma in children from lower socio-economic backgrounds.

Ocular biometry is also useful for other purposes. For example, measurements of the eye are made prior to cataract surgery to help determine the intraocular lens (IOL) power needed.

### OBJECT OF THE INVENTION

It is an object of the invention to provide an improved ocular biometry system and/or method that measures one or more eye parameters. Alternatively, it is an object to provide an improved ocular biometry system and/or method that goes at least some way to addressing the aforementioned problems. Alternatively, it is an object of the invention to at least provide the public with a useful choice.

### SUMMARY OF THE INVENTION

Aspects of the present invention are directed towards ocular biometry systems and methods for measuring parameters of an eye. More particularly, aspects of the present invention are directed towards measuring parameters of an eye by capturing images of the eye when at least one light source is shone into the eye, and determining the parameters of the eye by analysing the captured images.

According to one aspect of the invention, there is provided an ocular biometry system comprising:
a light source configured to generate a light beam for incidence on an eye;
first and second cameras configured to capture images of the eye when the light beam passes through the eye; and
one or more processors configured to:
   identify a plurality of features in the captured images, the plurality of features being representative of the light beam passing from one part of the eye to another part of the eye; and
   determine, from the identified plurality of features, one or more parameters of the eye.

Preferably the light source comprises a non-visible light source. More preferably the non-visible light source comprises an infra-red light source. In embodiments of the invention, the light source comprises a laser.

Preferably the parameters determined by the one or more processors are one or more of the parameters selected from the group consisting of: axial length; anterior chamber depth; posterior chamber depth; lens thickness; corneal radius/curvature; anterior lens radius/curvature; posterior lens radius/curvature; and retinal radius/curvature.

In some embodiments the plurality of features identified in the captured images are representative of the light beam passing from and to one or more of the parts of the eye selected from the group consisting of: cornea; anterior chamber (aqueous humour); posterior chamber; lens; vitreous humour; and retina.

In some embodiments of the invention, the system comprises a beam adjustment mechanism configured to adjust the light beam to be incident on the eye in a plurality of incidence positions, wherein the captured images comprise a plurality of images, each of the plurality of images being of the light beam passing through the eye when the light beam is in a different incidence position of the plurality of incidence positions, and wherein the one or more processors are configured to determine the parameters from each of the plurality of images.

In certain embodiments of the invention, the beam adjustment mechanism comprises a reflector, the light beam being reflected by the reflector before entering the eye, and a reflector adjustment mechanism configured to adjust the orientation and/or position of the reflector. The reflector may comprise a mirror. Alternatively, the reflector may comprise a prism.

Preferably, the one or more processors identify the plurality of features in the captured images by identifying regions of relatively high intensity light in the captured images, the regions of relatively high intensity light corresponding to the plurality of features.

Preferably, the one or more processors determine an optical path length between two locations in the eye from positions of the features in the captured images, and calculate a geometric path length between the two locations in the eye from the optical path length. The geometric path length may be representative of, or equivalent to, one of the parameters.

In some embodiments of the invention the light source comprises one or more light sources configured to generate the light beam, wherein the light beam is a first light beam for incidence on the eye, and the one or more light sources are further configured to generate a second light beam for incidence on the eye, the first and second light beams being separated by a distance when incident on the eye, and further wherein the first and second cameras are configured to capture images of the eye when the first and second light beams pass through the eye.

In some embodiments of the invention the one or more light sources comprise first and second light sources. Alternatively, the one or more light sources comprise a single light source and a beam splitter for generating the first and second light beams from the single light source. More preferably the first and second light sources comprise first and second non-visible light sources.

More preferably the first and second non-visible light sources comprises first and second infra-red light sources, for example lasers.

Preferably, the one or more light sources are configured such that the first and second light beams are incident on the eye symmetrically with respect to an axis of the eye.

Preferably, the beam adjustment mechanism is configured to adjust the first and second light beams to be incident on the eye in a plurality of incidence positions, wherein the captured images comprise a plurality of images, each of the plurality of images being of the first and/or second light beams passing through the eye when the first and second light beams are in different incidence positions of the plurality of incidence positions, and wherein the one or more processors are configured to determine the parameters from each of the plurality of images.

In some embodiments of the invention, the beam adjustment mechanism comprises a first beam adjustment mechanism configured to adjust the first light beam to be incident on the eye in a plurality of incidence positions and a second beam adjustment mechanism configured to adjust the second light beam to be incident on the eye in a plurality of incidence positions.

Preferably the ocular biometry system comprises third and fourth cameras configured to capture images of the eye when the first and second light beams pass through the eye. More preferably, the first and third cameras are positioned symmetrically relative to the eye and are configured to capture images of a first set of parts of the eye, and the second and fourth cameras are positioned symmetrically relative to the eye and are configured to capture images of a second set of parts of the eye.

According to another aspect of the invention, there is provided an ocular biometry system comprising:
a light source configured to generate a light beam for incidence on an eye;
first and second cameras configured to capture images of the eye when the light beam passes through the eye; and
one or more processors configured to:
   store the captured images in a memory, the captured images comprising a plurality of features, the plurality of features being representative of the light beam passing from one part of the eye to another part of the eye.

Preferably, the one or more processors are further configured to:
identify the plurality of features in the captured images; and
determine, from the identified plurality of features, one or more parameters.

In some embodiments the one or more processors comprises one or more first processors configured to store the captured imaged in the memory and one or more second processors configured to identify the plurality of features and determine the one or more parameters. The one or more second processors may be remote from the one or more first processors.

According to another aspect of the invention, there is provided a processor-implemented method of measuring a parameter of an eye, the method comprising:
receiving images of the eye when one or more light beams pass through the eye;
identifying a plurality of features in the images, the plurality of features being representative of the light beam passing from one part of the eye to another part of the eye; and
determining the parameter from the identified plurality of features.

Preferably the method comprises determining one or more parameters of the eye, the parameters being one or more of the parameters selected from the group consisting of: axial length; anterior chamber depth; posterior chamber depth; lens thickness; corneal radius/curvature; anterior lens radius/curvature; posterior lens radius/curvature; and retinal radius/curvature.

In some embodiments the plurality of features identified in the images are representative of the light beam passing from and to one or more of the parts of the eye selected from the group consisting of: cornea; anterior chamber (aqueous humour); posterior chamber; lens; vitreous humour; and retina.

Preferably, the method comprises identifying the plurality of features in the images by identifying regions of relatively high intensity light in the captured images, the regions of relatively high intensity light corresponding to the plurality of features.

Preferably, the method comprises determining an optical path length between two locations in the eye from positions of the features in the images, and calculating a geometric path length between the two locations in the eye from the optical path length. The geometric path length may be representative of, or equivalent to, one of the parameters.

Preferably, the method comprises:
controlling a beam adjustment mechanism to adjust the light beam to be incident on the eye in a plurality of incidence positions;
receiving the plurality of images, each of the plurality of images being of the light beam passing through the eye when the light beam is in a different incidence position of the plurality of incidence positions; and
determining the parameters from each of the plurality of images.

Preferably, the method comprises:
controlling a first beam adjustment mechanism to adjust a first light beam to be incident on the eye in a plurality of incidence positions; and
controlling a second beam adjustment mechanism to adjust a second light beam to be incident on the eye in a plurality of incidence positions.

Preferably, the method comprises controlling a reflector adjustment mechanism to adjust the orientation and/or position of a reflector, the light beam being reflected by the reflector before entering the eye.

According to another aspect of the invention, there is provided a processor-readable medium having stored thereon processor-executable instructions which, when executed by a processor, cause the processor to perform a method of measuring a parameter of an eye, the method comprising:
receiving a plurality of images of the eye when one or more light beams pass through the eye;
identifying a plurality of features in the images, the plurality of features being representative of the light beam passing from one part of the eye to another part of the eye; and
determining the parameter from the identified plurality of features.

According to another aspect of the invention, there is provided a method of measuring a parameter of an eye, the method comprising:
shining one or more light beams into the eye;
capturing a plurality of images of the eye when the light beam passes through the eye;
identifying a plurality of features in the captured images, the plurality of features being representative of the light beam passing from one part of the eye to another part of the eye; and
determining the parameter from the identified plurality of features.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

One or more embodiments of the invention will be described below by way of example only, and without intending to be limiting, with reference to the following drawings, in which:

FIG. 1 is a cross-sectional schematic illustration of an eye indicating anatomical terms of the eye;

FIG. 2 is a side view schematic illustration of an ocular biometry system according to an embodiment of the invention;

FIG. 3 is a side view schematic illustration of a control system according to an embodiment of the invention;

FIG. 4 is a flow chart of a method of measuring a parameter of an eye according to one embodiment of the invention;

FIG. 5 is a side view schematic illustration of part of the ocular biometry system shown in FIG. 2;

FIG. 6 is a side view schematic illustration of the ocular biometry system of FIG. 2;

FIGS. 7A and 7B are simplified sketched illustrations of images that may be captured by cameras in the ocular biometry system of FIG. 2;

FIG. 8 is a plan view schematic illustration of an ocular biometry system according to another embodiment of the invention; and

FIG. 9 is a side view schematic illustration of the ocular biometry system of FIG. 8.

### DETAILED DESCRIPTION OF PREFERRED EMBODIMENTS OF THE INVENTION

Embodiments of the present invention are directed towards ocular biometry systems and methods for measuring parameters of an eye, which may be referred to as eye biometrics. In the context of the present invention, “biometrics” are understood to mean measurements of the body. In general terms some embodiments of the invention involve measuring parameters of an eye by capturing images of the eye when at least one light source is shone into the eye. The parameters of the eye are determined from analysis of the captured images.

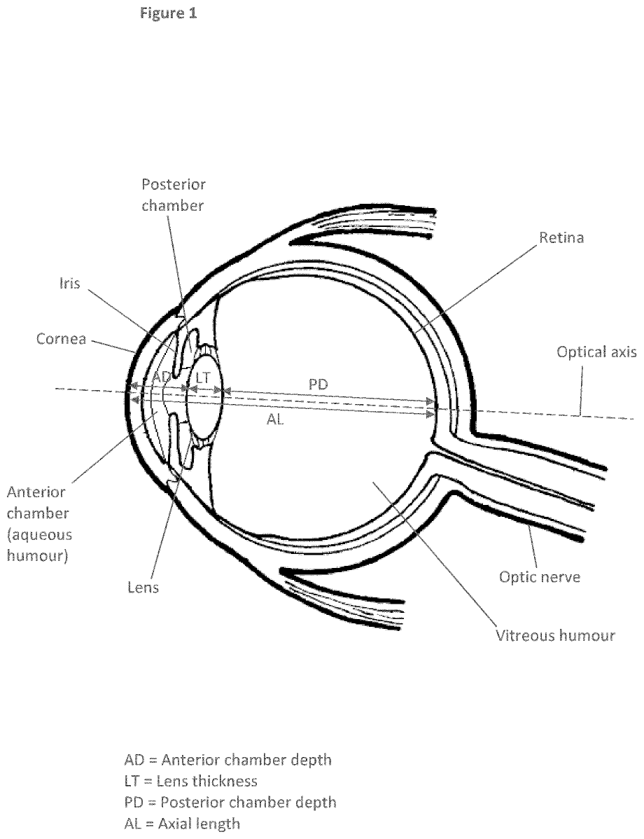

FIG. 1 is a cross-sectional schematic illustration of an eye indicating anatomical terms of the eye as referred to in this specification.

In the context of the specification, “light” will be understood to mean electromagnetic radiation, including visible and non-visible parts of the electromagnetic spectrum. Preferred forms of the present technology use “non-visible light”, i.e. those parts of the electromagnetic spectrum that cannot be seen by the eye being measured. If visible light is shone into the eye, the eye will typically adjust in some way to accommodate for the light, for example by altering the shape of the lens, so this may result in altered measurements of one or more parameters of the eye.

Ocular Biometry System

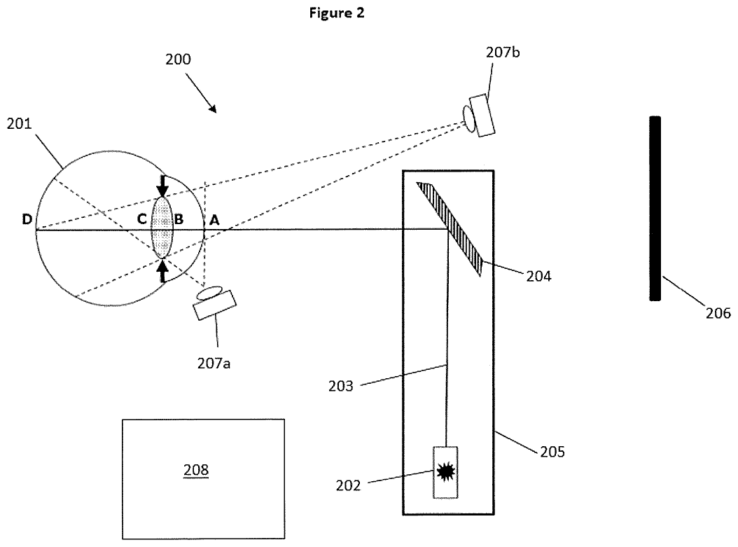

An ocular biometry system for measuring parameters of the eye is illustrated in FIG. 2, which is a side view schematic illustration of an ocular biometry system 200 according to an embodiment of the invention.

Ocular biometry system 200 comprises a light source 202 configured to generate a light beam 203 which is made incident on, i.e. shone into, eye 201. In the embodiment shown in FIG. 2, the light beam 203 initially has a path that is perpendicular to the optical axis of the eye, for example in the superior or inferior direction in relation to the patient, and is reflected before entering the eye by reflector 204. Reflector 204 may take the form of a mirror or a reflecting prism positioned to reflect the light beam 203 towards the eye 201. In other embodiments of the invention, reflector 204 may comprise a system of mirrors and/or prisms. In some embodiments, the light beam 203 may pass through an optical arrangement that helps maintain the optical properties of the light beam following reflections may be provided, for example parallel mirrors. In another embodiment of the invention, light source 202 may be positioned to project light beam 203 directly along the optical axis of the eye. It will be appreciated by the skilled addressee that the alternative configurations described in this (and other) paragraphs may also be applied to embodiments of the invention described and illustrated elsewhere in this specification.

In the embodiment of the technology described here, light source 202 is a source of non-visible light and light beam 203 is a beam of non-visible light. For example, light source 202 may be an infra-red laser. As explained above, one reason to use non-visible light in system 200 is to avoid the eye 201 adjusting to accommodate for the light, for example by altering the shape of the lens, which may result in altered measurements of one or more parameters of the eye.

The ocular biometry system 200 may further comprise other optical components acting on the light beam 203 before being incident on the eye 201. In some embodiments, optical components configured to reduce the width of the light beam 203, for example an opaque member comprising pinholes configured to transmit a portion of light beam 203, may be provided.

Light source 202, reflector 204 and any other optical components provided in system 200 may be housed in a housing 205. To measure parameters of an eye 201, the patient is stationed in front of housing 205. Ocular biometry system 200 may comprise one or more eye positioning mechanisms, for example a chin rest and forehead support, to enable the patient to position themselves and their eye in the desired position with stability and in comfort.

The system may further comprise a sight 206 for the patient to look at during use of system 200. The sight 206 may be positioned optically far from the patient so that the eye 201 accommodates to viewing into the distance. A system of mirrors may be used to position sight 206 optically far from, but geometrically (i.e. physically) close to, the patient, for example if it is not possible to position sight 206 geometrically (i.e. physically) far from the patient. In other implementations the sight 206 may be positioned another distance from the patient, e.g. optically closer to the patient if it is desirable to measure parameters of the eye with the eye in a particular accommodation configuration.

The ocular biometry system 200 shown in FIG. 2 further comprises first camera 207a and second camera 207b. The cameras 207 are positioned, and are configured, in a manner suitable to image the eye. In the embodiment of FIG. 2, first camera 207a is positioned to image generally anterior parts of eye 201, for example the cornea and lens, and second camera 207b is positioned to image at least a posterior part of eye 201, for example the cornea, lens and retina. First camera 207a is positioned inferior to the eye 201. Second camera 207b is positioned superior to the eye 201 and further away from the eye than first camera 207a. The cameras may be put in other positions in other embodiments.

In this specification the term “camera” refers to any image capturing device or system. It will be understood that the cameras 207 used are configured to capture images in the part of the electromagnetic spectrum corresponding to the light source 202, e.g. infra-red. Cameras used in embodiments of the invention may be still frame or continuously filming cameras. Further, when this specification refers to capturing an image it will be understood that the image may be obtained in digital form, i.e. the image may be represented by digital image data. Reference to an “image” in this specification will be understood to refer either to the visual representation of what is imaged or to the data that is representative of the image, or both.

In preferred embodiments of the invention, cameras 207 are stereo cameras with focal lengths selected to obtain clear images of the eye based on the typical size of the human eye and the distance of the camera from the eye.

While the system 200 in the embodiment of FIG. 2 comprises two cameras 207, other embodiments may use more than two cameras, for example four cameras. A greater number of cameras may improve the ability of the ocular biometry system 200 to accommodate small movements of the eye during imaging. Even if a patient keeps their eyes fixated on a specific point, uncontrollable eye movements can still occur. Fewer cameras means the patient needs to keep their vision fixed and their eye still to avoid measurement errors. A greater number of cameras enables small eye movements to be tolerated because images from all the cameras can be averaged. It will be appreciated that this principle applies to all embodiments described in this specification, even if not explicitly stated, and that embodiments of the invention may have any number of cameras.

In one example of a four-camera arrangement of the system 200 in FIG. 2, two cameras may be placed in a similar position as first camera 207a as shown in FIG. 2, with each camera being located either side of the vertical plane of symmetry of the eye, and two cameras may be placed similarly near the position of second camera 207b as shown in FIG. 2.

Ocular biometry system 200 may comprise one or more camera adjustment mechanisms configured to adjust the positions and/or orientations of cameras 207. For example cameras 207 may be mounted on camera mounts able to move and rotate relative to the eye 201.

Ocular biometry system 200 also comprises a control system 208. Control system 208 is configured to communicate with other components of system 200, including cameras 207, light source 202, reflector 204 and a beam adjustment mechanism(s) (not shown in FIG. 2 but described below). Control system 208 may receive data from, and send data to, other components of system 200, and/or external systems, and may control the operation of these components, for example the positioning/orientation of the components and/or their activation/deactivation.

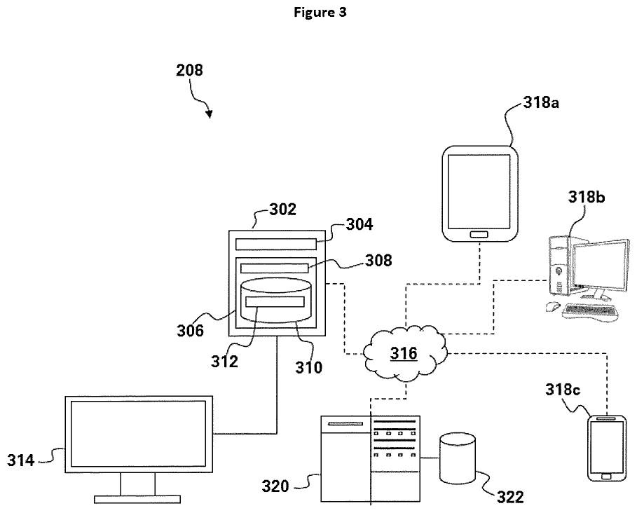

Control system 208 is shown in more detail in FIG. 3, which is a schematic illustration of a control system 208 according to an embodiment of the invention. Control system 208 comprises a local hardware platform 302 that manages the collection and processing of data relating to operation of the ocular biometry system 200. The hardware platform 302 has a processor 304, memory 306, and other components typically present in such computing devices. In the exemplary embodiment illustrated the memory 306 stores information accessible by processor 304, the information including instructions 308 that may be executed by the processor 304 and data 310 that may be retrieved, manipulated or stored by the processor 304. The memory 306 may be of any suitable means known in the art, capable of storing information in a manner accessible by the processor 304, including a computer-readable medium, or other medium that stores data that may be read with the aid of an electronic device.

The processor 304 may be any suitable device known to a person skilled in the art. Although the processor 304 and memory 306 are illustrated as being within a single unit, it should be appreciated that this is not intended to be limiting, and that the functionality of each as herein described may be performed by multiple processors and memories, that may or may not be remote from each other or from the ocular biometry system 200. The instructions 308 may include any set of instructions suitable for execution by the processor 304. For example, the instructions 308 may be stored as computer code on the computer-readable medium. The instructions may be stored in any suitable computer language or format. Data 310 may be retrieved, stored or modified by processor 304 in accordance with the instructions 310. The data 310 may also be formatted in any suitable computer readable format. Again, while the data is illustrated as being contained at a single location, it should be appreciated that this is not intended to be limiting—the data may be stored in multiple memories or locations. The data 310 may also include a record 312 of control routines for aspects of the system 300.

The hardware platform 302 may communicate with a display device 314 to display the results of processing of the data. The hardware platform 302 may communicate over a network 316 with user devices (for example, a tablet computer 318a, a personal computer 318b, or a smartphone 318c), or one or more server devices 320 having associated memory 322 for the storage and processing of data collected by the local hardware platform 302. It should be appreciated that the server 320 and memory 322 may take any suitable form known in the art, for example a “cloud-based” distributed server architecture. The network 316 may comprise various configurations and protocols including the Internet, intranets, virtual private networks, wide area networks, local networks, private networks using communication protocols proprietary to one or more companies, whether wired or wireless, or a combination thereof.

Linear/Axial Measurements

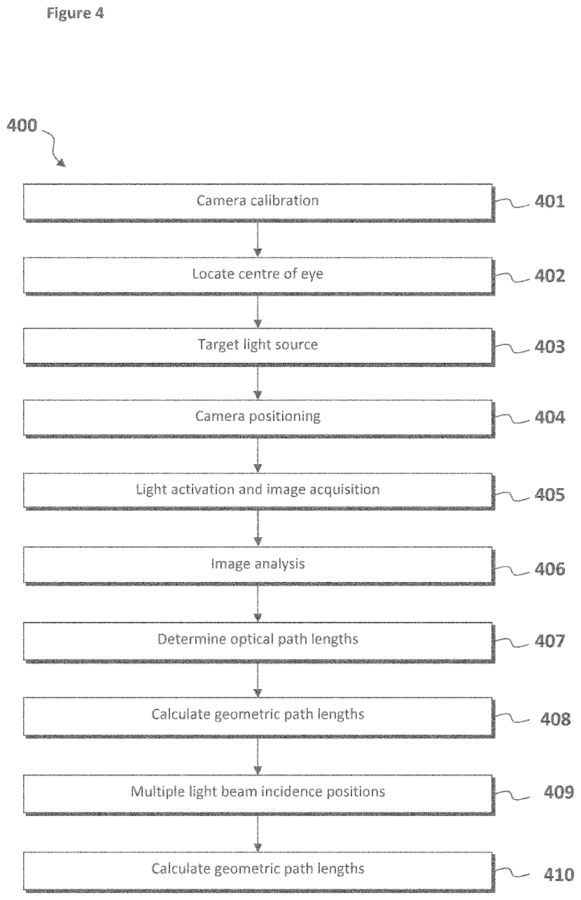

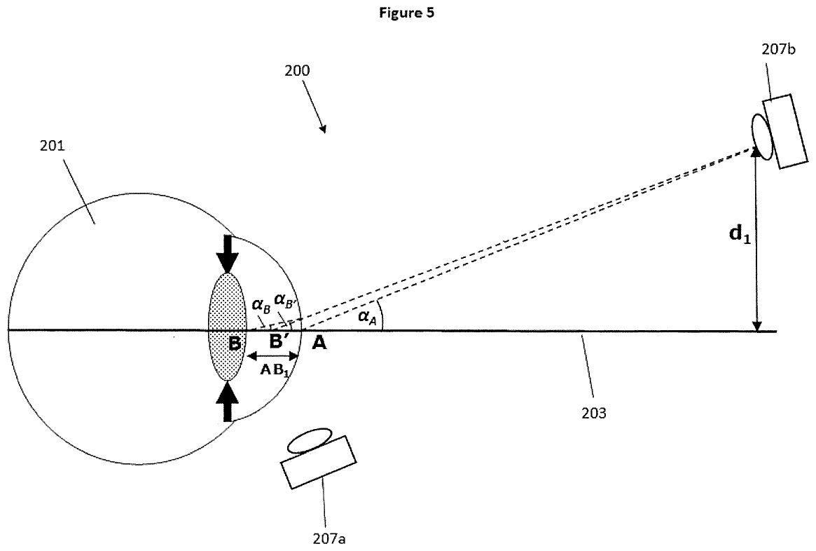

A method for measuring parameters of the eye will first be described with reference to FIG. 4, which is a flow chart of a method 700 of measuring a parameter of an eye according to one embodiment of the invention. The parameters of the eye 201 measured using a first part of this method are linear or one-dimensional measurements, i.e. distances within the eye along a single line or axis, for example along the optical axis of the eye. It will subsequently be described how method 400 may be extended to measure parameters of the eye along multiple lines/axes.

Calibration and Set Up

In step 401 the cameras 207 are calibrated. Any appropriate calibration technique may be used, for example positioning heat resistant material on which is printed a pattern in front of the eye 201 and imaging the heat resistant material with the cameras 207. An exemplary technique is explained here: Gschwandtner M, Kwitt R, Uhl A, Pree W, Infrared camera calibration for dense depth map construction, Intelligent Vehicles Symposium (IV), 2011 IEEE 2011 Jun. 5 (pp. 857-862).

In step 402 the centre of the eye 201 is located. Any suitable technique to locate the centre of eye 201 may be used. In certain embodiments of the invention, one of the cameras 207 is positioned directly in front of eye 201 on (or as close as possible to) the optical axis of eye 201 and an image of the eye, including the iris, is captured by the camera. A circle detection method is performed on the captured image to identify the iris in the captured image, and a circle centre location method is performed to locate the centre of the circle, which is assumed to correspond to the centre of eye 201 (i.e. the optical axis).

If an ocular biometry system 200 such as illustrated in FIG. 2 is used, reflector 204 may be a one-way mirror so that a camera 207 can be positioned on the optical axis of the eye 201 and image the eye 201 through the mirror. In other embodiments the camera may be positioned so that the reflector is not in its field of view, for example the camera is positioned slightly to the side of, or slightly above or below, the reflector. It will be appreciated that a relatively small mirror will enable the camera to be as close to the optical axis of the eye as possible in such an embodiment.

In step 403 the light source is targeted at the centre of the eye so that the light beam 403 is incident as closely along the optical axis of the eye 201 as possible. The ocular biometry system 200 comprises a beam adjustment mechanism configured to adjust the incidence of the light beam 203 on the eye 201. The beam adjustment mechanism may comprise one or more mechanisms to adjust the position and/or orientation of components of the ocular biometry system 200. For example the beam adjustment mechanism may comprise a mechanism for moving housing 205. In one embodiment, with the eye 201 in position, the housing 205 is coarsely adjusted so that the reflector 204 is generally at eye level. The light source 202 is activated so that light beam 203 is incident on the eye 201. In the embodiment shown, light beam 203 reflects off reflector 204 before entering the eye. The beam adjustment mechanism may further comprise one or more mechanisms for adjusting the position and/or orientation of the light source 202 and/or the reflector 204 in order to adjust the light beam 203 to be incident on the centre of the eye 201, for example as a fine adjustment step after the coarse adjustment of the housing 205.

The patient may be asked to look at sight 206 during this process so that the eye 201 accommodates to viewing into the distance.

In step 404 the cameras 207 are positioned to capture images of the eye 201. In certain embodiments the cameras 207 are moved such that camera 207a is positioned inferior to the eye 201 to image generally anterior parts of eye 201, for example the cornea and lens, and camera 207b is positioned superior to the eye 201 to image multiple parts of eye 201, for example the cornea, lens and retina, as shown in FIG. 2. In other embodiments the cameras may be located in other positions relative to the eye. Camera properties, such as zoom, shutter speed, aperture and ISO, may also be configured during step 404. Information on the field of view of each camera may be provided to the control system 208 during step 404.

The calibration and set up steps described above may not be required in all exemplary methods. For example, methods according to embodiments of the invention may be performed using an ocular biometry system already configured to perform said methods.

Imaging

In step 405 light source 202 is activated and light beam 203 is shone into eye 201, such as is shown in FIG. 2. While light beam 203 is shining into eye 201, cameras 207 capture images of the eye 201 (i.e. those parts of the eye within the field of view of each camera).

In some embodiments the captured images are transmitted from the camera to control system 208. It will be appreciated that any suitable method of transmission may be used, including wireless or wired data transmission. Auxiliary image information may also be provided from the camera to control system 208, either contemporaneously with or subsequently to the sending of the captured images. Auxiliary image information may be additional information related to the captured images, for example properties of the camera taking an image (e.g. make, model, shutter speed, focal length, aperture settings, ISO, etc), the location of the camera in the system or any other information that may be required or useful to analyse the captured images.

In embodiments of the invention the control system 208 limits the time of activation of the light source 202 (or light sources in embodiments with multiple light sources, such as described below). The maximum exposure time of light to the patient depends on the type of light generated by the light source and the time of activation is limited based on the maximum exposure time in order to ensure patients are exposed to safe amounts of light.

Image Analysis

On receipt of the captured images the control system 208 may store the captured images in memory 306 for immediate processing or for processing at a later time. Alternatively, the control system 208 may send the captured images to a remote memory, for example memory 322 via server 320, for processing at a later time.

In step 406 the captured images are analysed by the one or more processors 304. In FIG. 3 processor 304 is illustrated as forming part of local hardware platform 302. In other embodiments multiple processors 304 may be provided, including one or more processors remote from the ocular biometry system 200. In other embodiments all of the processors are located remote from the ocular biometry system 200.

In some embodiments the processor 304 may perform one or more image pre-processing steps on the captured images, for example noise reduction or averaging of images from multiple cameras to lessen the effects of small eye movements on the analysis.

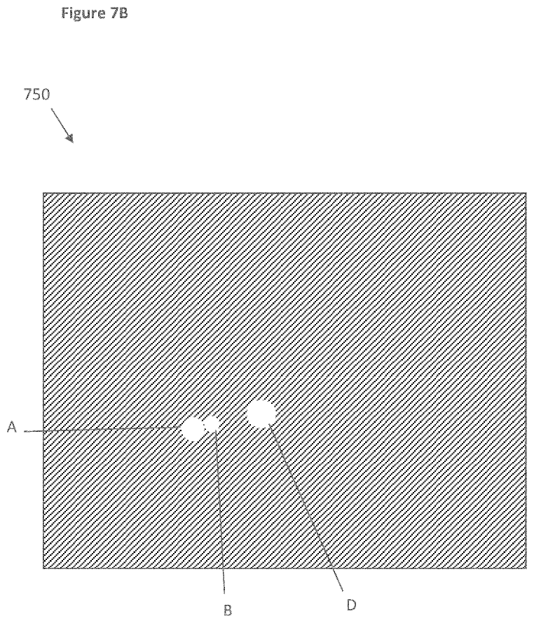

FIGS. 7A and 7B are simplified sketched illustrations of images that may be captured by cameras 207a and 207b in the ocular biometry system 200 of FIG. 2. In this embodiment, image 700 (FIG. 7A) is captured by camera 207a positioned inferior to the eye 201 with the cornea and lens in its field of view, and image 750 (FIG. 7B) is captured by camera 207b positioned superior to the eye 201 with the cornea, lens and retina in its field of view. The shaded regions in images 700 and 750 are regions of low intensity light while the non-shaded regions are regions of relatively high intensity light.

In certain embodiments of the invention, the processor 304 is configured to analyse images 700 and 750 to identify features in the images that are representative of light beam 203 passing from one part of the eye to another part of the eye. In the embodiment presently described, the features correspond to regions of relatively high intensity light in images 700 and 750 and the processor 304 identifies the regions of relatively high intensity light using conventional image analysis techniques.

When light beam 203 passes through the eye 201 it passes through the cornea, anterior chamber (aqueous humour), lens, vitreous humour and is incident on the retina. Along this path the beam 203 passes from one medium to another in several locations, marked as A, B, C and D in FIG. 2:
1. A (cornea surface): the beam 203 passes from air into the cornea;
2. B (anterior lens surface): the beam 203 passes from the aqueous humour into the lens;
3. C (posterior lens surface): the beam 203 passes from the lens into the vitreous humour; and
4. D (retina surface): the beam 203 is incident on the retina.

At each of these points the beam 203 is refracted and partly reflected. This causes a scattering of some of the light in beam 203, which is seen by cameras 207 as a ‘halo’ or region of higher intensity light compared to other parts of the field of view.

In other embodiments other points in the eye 201 may also be identified through features in the captured images, for example the posterior chamber and iris.

In images 700 and 750 the regions of higher intensity light labelled A, B, C and D correspond to the locations A, B, C and D within the eye 201 shown in FIG. 2 and that are described above as the locations in the eye 201 that the light beam is scattered from. The processor 304 is configured to identify which regions of high intensity light in the images correspond to which locations within the eye 201 based on their location within the images 700 and 750 and the field of view of the respective cameras 207a and 207b.

If the camera is further away from the nose compared to the laser (i.e. the camera is temporal), then the region of higher light intensity closest to the camera (temporal) corresponds to the posterior eye (i.e. retina). If the camera is closer to the nose compared to the laser (i.e. the camera is nasal), then the point closest to the camera (nasal) corresponds to the anterior eye (i.e. cornea).

In image 700 the furthest temporal high intensity region (i.e. away from the nose) corresponds to the corneal surface reflection point A. The subsequent high intensity regions are arranged in a straight line in the image away from the region corresponding to corneal reflection point A, with the correspondence occurring in the order of the reflection points as the beam 203 passes into the eye 201, i.e. in the order A then B then C then D. The number of these reflection points that appear in each of images 700 and 750 depends on the field of view of the cameras 207 capturing the respective image.

Therefore, in image 700 (captured by inferior camera 207a) the high intensity region further from the nose (the left-most region in image 700) is identified by the processor 304 as corresponding to the corneal surface reflection point A, while regions B and C are recognised as corresponding to the anterior lenticular surface reflection point B and the posterior lenticular surface reflection point C respectively. Since the part of the retina on which beam 203 is incident is not in the field of view of camera 207a there is no high intensity light region corresponding to the retinal surface reflection point D in image 700.

Image 700 includes another high intensity light region R. It has been found that minor reflections within the eye can lead to other regions of high intensity in the images captured by cameras 207. Such regions may be identified by the processor 304 as not corresponding to important reflection points within the eye if they do not lie on the same straight line as the other high intensity regions in the image, as is the case with region R in image 700. In some embodiments, the processor 304 is therefore configured to ignore high intensity regions not on a straight line with the other regions in an image.

In image 750 (captured by superior camera 207b) the high intensity region furthest from the nose (the left-most region in image 750) is recognised by the processor 304 as corresponding to the corneal surface reflection point A, while regions B and D are recognised as corresponding to the anterior lenticular surface reflection point B and the retinal reflection point D respectively. It has been found that the posterior lenticular surface reflection point C does not appear in an image captured by a camera in the position of camera 207b in the embodiment of FIG. 2, or is hidden by the light from point B. In preferred embodiments of the invention at least two reflection locations in the eye 201 are captured in images in both cameras 207. The position of the two reflection locations may then be identified in images from both cameras and correlated spatially, enabling the positions of all other reflection locations captured in the images to be determined from their position relative to the correlated reflection locations.

The positions of the high intensity light regions A, B, C and D in images 700 and 750 are analysed using conventional image feature recognition techniques. For example, in the embodiment of the invention resulting in images 700 and 750, the high intensity regions are generally circular and circle detection techniques are used to identify the position of the centre of each circle within the image. Co-ordinates are allocated by the processor 304 to each of the regions.

In step 406 it will be understood that, when identifying features in an image, the processor 304 may operate by identifying such features from the image data representative of the image. In some embodiments it may not be necessary for the processor 304 to first construct the visual representation of the image from the image data in order to be able to identify the features.

Alternatively, or additionally, the processor 304 may be configured to construct a visual representation of the image from the image data and identify the features from the visual representation.

Biometry Calculations

In step 407 the processor 304 determines the optical path length (OPL) between two or more locations in eye 201 from the positions of the features in the captured images. In the case of images 700 and 750 the processor determines the apparent positions of any two or more of locations A, B, C and D and the apparent distance between those locations (OPL) in eye 201 are also calculated. Those skilled in the art will appreciate how to determine the OPLs from detection of the positions of the regions A, B, C and/or D in the images and calibration information using conventional techniques. In one example, image thresholding techniques may be used.

The OPL between any two points A, B, C and D may differ from the geometric path length (GPL) between the same points because of refraction in the different media in the eye distorting the path of light beam 203 and the path of the light reflected from the reflection points and captured by the cameras 207. In step 408 the processor 304 calculates the GPL(s) between two or more locations in eye 201 from the corresponding calculated OPL(s).

Two exemplary methods are described here for calculating one or more GPLs from the OPL(s) determined from the captured images. Other methods may be used in other embodiments of the invention.

Method 1: Apparent Depth Calculation Generally speaking the method performed by the processor 304 in this embodiment applies Snell's law at each boundary at which the light beam passes between two different media within the eye to correct the optical distortion seen by the cameras 207.

Since point A is the corneal surface, the interface is an air/cornea interface and the optical and geometric positions of point A are the same.

To determine the geometric position of point B (the anterior lenticular surface), Snell's law is applied. This will now be described with reference to FIG. 5, which is a side view schematic illustration of part of the ocular biometry system 200 shown in FIG. 2.

According to Snell's law for the light captured by superior camera 207b:

nₐᵢᵣ·sin α_{A}=n_{aqueous}·sin α_{B′}

where nₐᵢᵣ is the refractive index of air, n_{aqueous} is the refractive index of the aqueous humour and angles α_{A} and α_{B′} are, respectively, the angle between the incident light beam 203 and the light received by camera 207b reflected from the surface of the cornea (point A) and the apparent angle between the incident light beam 203 and the light received by camera 207b reflected from the anterior surface of the lens (point B), i.e. virtual point B′.

For small angles as per the configuration in FIG. 5, sin α_{A}=tan α_{A},

therefore: n_{aqueous}·OPL_{AB1}≈AB₁

where OPL_{AB1} is the optical path length between A and B as seen by superior camera 207b (i.e. AB′, the distance between A and B′ as shown in FIG. 5) and AB₁ is the geometric path length between A and B.

A similar calculation can be made for the light captured by inferior camera 207a. For the sake of clarity in the figure the distances and angles have not been marked in FIG. 5 but they will be understood to correspond to those for camera 207b to give:

n_{aqueous}·OPL_{AB2}≈AB₂

where OPL_{AB2} is the optical path length between A and B as seen by inferior camera 207a and AB is the geometric path length between A and B.

Clearly the geometric path length must be the same in both cases:

AB₁=AB₂.

The linking ratio V between the optical path lengths according to superior camera 207b and inferior camera 207a may be written as the tensor convolution:

φ=OPL_{AB1}⊗OPL_{AB2}.

Having effectively undistorted reflection point B, the geometric path lengths BC and BD may be calculated using a similar calculation.

Finally, the geometric path lengths AD and CD may be calculated by repeating the above steps at each reflection point (B, C and D) and replacing these points in the above equation. These steps can be performed at each step independently, undistorting A, B, C and D points one at a time. Alternatively, matrices of the incident (SC₁) and refraction (SC₂) angles at all the points (A, B, C and D) can be created, and all OPLs can be estimated from a convolution of these matrices:

OPL=SC₁⊗SC₂

Then, the GPLs (physical distances between points A, B, C and D) may be determined together, by the summation/integration of all the OPLs convoluted with the refractive indices of media of the eye (i.e. aqueous humour, lens and vitreous humour):

$$G P L = {\sum }_{D}^{i = A} OP L ⊗ {N}_{i}$$

Where the GPL is a matrix in the form:

$$[ \begin{matrix}\overline{AB} \\ \overline{BC} \\ \overline{CD} \\ \overline{AD}\end{matrix} ]$$

Method 2: Correlation Function

In an alternative embodiment a secondary modality is used to undistort the optical path lengths between points A, B, C and D determined from the images captured by cameras 207. In this embodiment, the method 400 is performed on a number of test subject eyes and the optical path lengths between one or more of points A, B, C and D are determined from the captured images. The same parameters are measured for the same test subject eyes with an alternative measuring technique, including but not limited to magnetic resonance imaging (MRI), ultrasound, interferometry (e.g. using the Lenstar™ or IOLMaster™ devices), for example.

If measurements are made for a sufficiently large sample of test subject eyes then a correlation function between geometric path lengths determined by the alternative eye biometric/parameter measurement method and the optical path lengths determined by the method 700 may be determined. This correlation function may subsequently be used to calculate geometric path lengths between two locations in an eye corresponding to optical path lengths determined by method 400.

The result of either method 1 or method 2 as described above is one or more geometric path lengths between locations in eye 201, the geometric path lengths being parameters of the eye/biometrics. In the above described example, in which the distances between locations A, B, C and D (as labelled in FIG. 2) are determined, these parameters are:
AB: Anterior chamber depth;
BC: Lens thickness;
CD: Posterior chamber depth; and
AD: Axial length.

Once calculated, the determined parameters may be stored in memory 306 or memory 322, output via display device 314 or communicated to other devices, for example over network 316. In preferred embodiments the parameters are used to assess refractive errors in the patient.

Multiple Light Beam Incidence Positions in some embodiments of the invention additional parameters of the eye may be determined by shining the light beam 203 into the eye 201 in a number of incidence positions and determining parameters for each incidence position. This enables parameters of the eye to be determined in multiple locations within the eye. In some embodiments parameters of the eye are determined at multiple locations along an axis of the eye in a first direction, for example the inferior-superior or lateral directions. In this way a two-dimensional model of parts of the eye along an axis can be generated. In some embodiments parameters of the eye are additionally determined along a second axis of the eye in a second direction. In this way a three-dimensional model of parts of the eye can be generated in the manner of a raster scan of the eye using the plurality of light beam incidence positions.

To achieve multiple light beam incidence positions, in some embodiments the ocular biometry system comprises a beam adjustment mechanism to adjust the light beam 203 to be incident on eye 201 in a plurality of incidence positions. Exemplary beam adjustment mechanisms have been described above in relation to targeting the light beam 203 at the centre of eye 201 in step 403. The same or similar beam adjustment mechanisms may be used to achieve multiple light beam incidence positions during the image acquisition process.

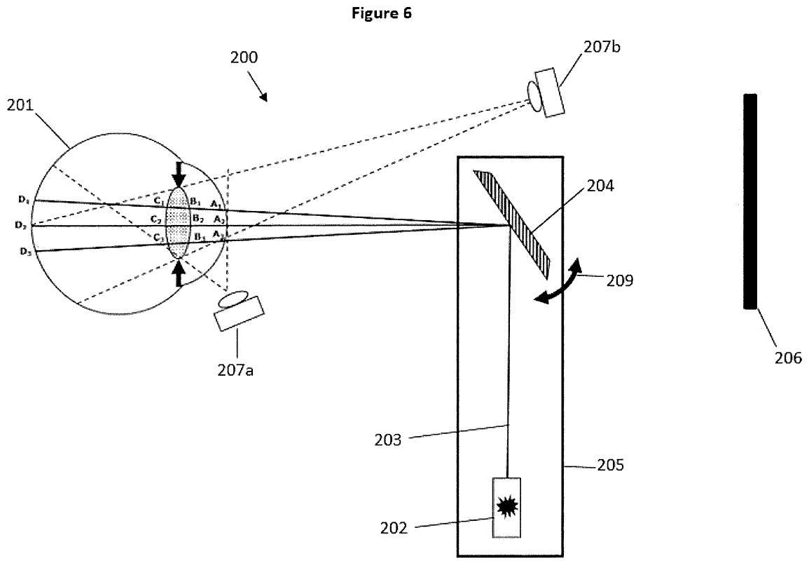

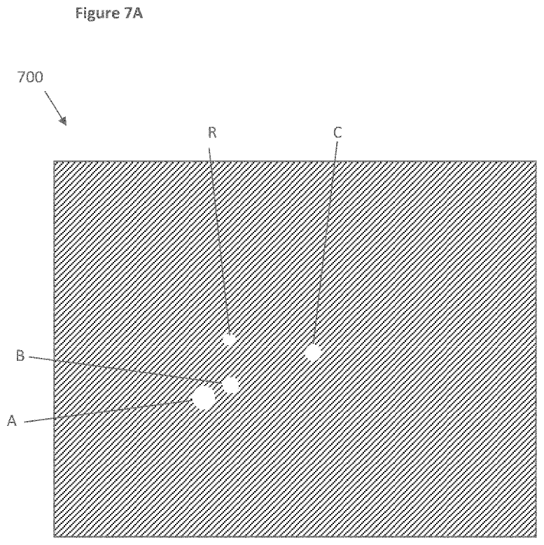

In step 409 the control system 208 controls the beam adjustment mechanism to adjust the light beam to be incident on the eye in a plurality of incidence positions. In the embodiment shown in FIG. 6, which is a side view schematic view illustration of ocular biometry system 200 according to one embodiment of the invention, the beam adjustment mechanism comprises a reflector adjustment mechanism configured to adjust the orientation of reflector 204, as shown by arrow 209 in FIG. 6. In the embodiment shown the reflector adjustment mechanism rotates reflector 204 around a horizontal axis, adjusting the incidence of light beam 203 on eye 201 in a vertical plane. In another embodiment the reflector adjustment mechanism rotates reflector 204 around a vertical axis, adjusting the incidence of light beam 203 on eye 201 in a horizontal plane. In other embodiments the reflector may be rotated around an axis in another direction, or the reflector adjustment mechanism may be configured to rotate the reflector around a plurality of axes, for example vertical and horizontal axes. In other embodiments the reflector adjustment mechanism is configured to adjust the position of the reflector 204 in addition to, or instead of adjusting the orientation of reflector 204. In still further embodiments, the beam adjustment mechanism comprises a mechanism for adjusting the position and/or orientation of light source 202.

In certain embodiments, the system 200 may comprise a lens or other optical component configured to cause all light beams incident on eye 201 to travel in parallel. For example, the light beams may all be parallel to the optical axis of the eye. This may be achieved in one embodiment by locating a lens between reflector 204 and eye 201 with the point of reflection of the light beam from the reflector 204 being the focal point of the lens. In another embodiment two reflectors may be used, with the focal point of the lens being located between the reflectors. In other embodiments, another afocal arrangement of optical components acting on incident light may be provided. Such arrangements may be advantageous to ensure the optical properties of the light beam entering the eye are the same as those of the light beam generated by the light source.

Cameras 207 capture images of the light beam 203 passing through eye 201 for each of the plurality of light beam incidence positions.

In some embodiments the control system 208 controls activation of the light source 202 such that the light source repeatedly turns on and off, and control system 208 further controls the beam adjustment mechanism to adjust the components of the system (e.g. the orientation of reflector 204) that determine the incidence position of the light beam 203 while the light source 202 is turned off. Once the beam adjustment mechanism has suitably adjusted reflector 204, for example, the control system 208 re-activates the light source 202. This sequence is repeated for the number of incidence positions are required. In this manner the total exposure time of the eye 201 to the light source can be reduced to improve safety.

In an alternative embodiment the ocular biometry system comprises a shutter configured to selectively block the light beam from the light source. Control system 208 is configured to control the shutter to expose the eye to the light beam once the beam adjustment mechanism has adjusted the components of the system as required. The shutter may be controlled by selectively moving it between a first position in which it blocks the light beam from the light source and a second position in which it does not block the light beam from the light source, for example.

The number of incidence positions, and therefore the number of captured images of the light beam 203 passing through eye 201, and the spacing between incidence positions, may be selected depending on the parameters of the eye desired to be obtained. In one embodiment a sufficient number of incidence positions are provided such that light beam 203 is incident across a sector of the retina subtending an angle of substantially 60° as parameters across such a range may be particularly clinically useful in some circumstances. For example this ensures parameters are determined for parts of the retina including the macula and blind spot.

In step 410 the geometric path lengths/eye parameters are calculated by processor 304 for each of the light beam incidence positions. This step comprises similar methods to those described above in relation to steps 406, 407 and 408 applied to each of the images captured by cameras 207 for each light beam incidence position. The result of this step, referring to the labelling in FIG. 6 are positions of, or distances between, the points A_{1 . . . n}, B_{1 . . . n}, C_{1 . . . n} and D_{1 . . . n} where n is the number of incident positions of light beam 203. Processor 304 may use this information to generate a two-dimensional model of parts of eye 201, or three-dimensional if the incidence positions vary in more than one plane. Such embodiments may enable determination of eye parameters such as axial length, anterior chamber depth, posterior chamber depth, and lens thickness in multiple locations within the eye, thus enabling further parameters to be determined, for example corneal radius/curvature, anterior lens radius/curvature, posterior lens radius/curvature, and retinal radius/curvature. In embodiments in which the lens depth is determined for multiple incidence positions, a two- or three-dimensional model of the lens and the change in shape of the lens away from the optic axis of the eye may be generated by processor 304. This may be useful for measuring astigmatisms.

Increasing the number of incidence positions may increase the accuracy of the calculated parameters of the eye but may lengthen the duration of the scan (period of taking measurements) and the time and complexity of calculations.

Multiple Light Sources

In some embodiments of the invention multiple light sources are shone into the eye. Such embodiments may collect more information on the structure of the eye.

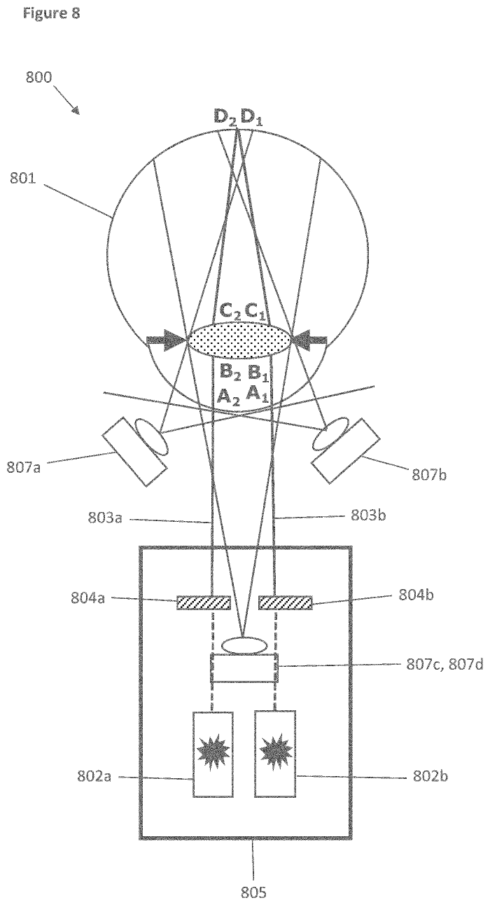

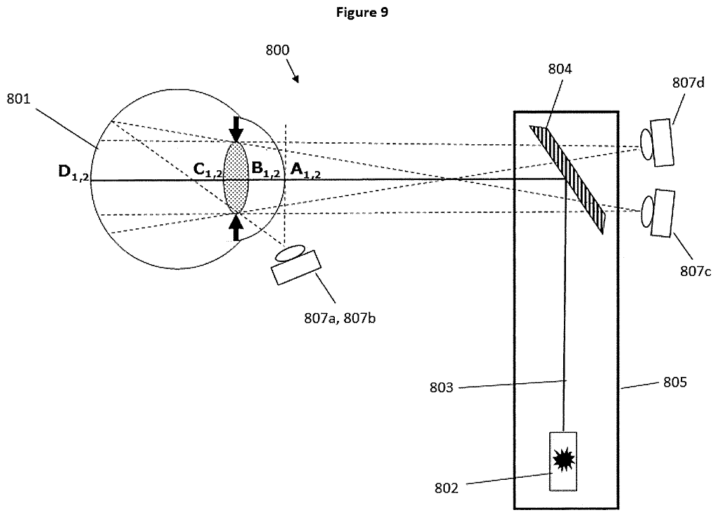

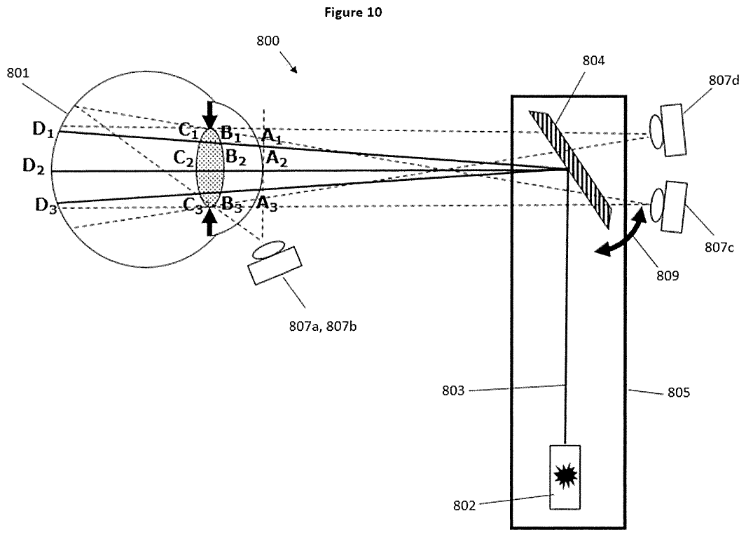

An exemplary embodiment is shown in FIGS. 8 and 9, which are plan and side view schematic illustrations of an ocular biometry system 800 according to another embodiment of the invention. Features of ocular biometry system 800 are similar to those in ocular biometry system 200 described above. It should be understood that features or modifications of the components in system 200 as described above may also apply to system 800 unless specifically stated otherwise.

Ocular biometry system comprises two light sources 802a and 802b positioned laterally next to each other from the perspective of the patient whose eye 801 is being measured when standing in front of ocular biometry system 800. Light sources 802a and 802b project light beams 803a and 803b respectively incident on eye 801. Similarly to the configuration of system 200, each of light beams 803a and 803b are initially projected in the superior direction and are reflected off reflectors 804a and 804b respectively before entering the eye 801 (light sources 802a and 802b are shown in FIG. 8 despite being vertically positioned below reflectors 804, as shown in FIG. 9, for illustrative purposes). Again, other configurations may be employed in alternative embodiments.

In the embodiment of FIGS. 8 and 9, two light sources 802a and 802b are provided. The light sources 802 may produce the same form of light, e.g. visible, non-visible, infra-red, while in other embodiments the light sources produce different forms of light.

In an alternative embodiment a single light source is provided and the ocular biometry system comprises a beam splitter to split the light beam from the single light source into two light beams for incidence on the eye, and reflectors to reflect the split light beams in parallel towards the eye. This avoids the expense of two light sources.

When incident on eye 801 the lights beams 803a and 803b are spaced apart by a distance. In the embodiment shown in FIG. 8 the light beams 803a and 803b are laterally spaced apart with respect to the patient and in the same horizontal plane. In other embodiments of the invention the lights beams are spaced apart in a different direction, for example in the same vertical plane and spaced apart in the superior-inferior direction relative to the patient. In certain embodiments of the invention the light sources and/or reflectors are configured such that the light beams 803a and 803b are incident on eye 801 symmetrically with respect to an axis of the eye, for example the optical axis.

The light sources 802, reflectors 804 and, if present, beam splitter, may be housed in a housing 805.

Ocular biometry system 800 further comprises a plurality of cameras 807 configured to capture images of the eye 801 when the first and second light beams 803 pass through the eye. At least two cameras 807 are provided. In the case of two cameras 807, they are positioned in a similar manner to the cameras 207 as described in relation to the embodiment shown in FIG. 2.

In the embodiment of FIGS. 8 and 9 four cameras 807 are provided. As explained above, fewer or more cameras may be provided in other embodiments. Two of the cameras are positioned to image generally anterior parts of eye 801, for example the cornea and lens, and two of the cameras are positioned to image at least a posterior part of eye 801, for example the cornea, lens and retina. In the embodiment shown cameras 807a and 807b are positioned symmetrically relative to the eye. For example, cameras 807a and 807b are positioned inferior to the eye 801 and laterally symmetric to the eye on the same horizontal plane as each other. Cameras 807a and 807b are relatively proximate to the eye compared to cameras 807c and 807d, which are also positioned symmetrically relative to the eye. In the example shown cameras 807c and 807d are positioned respectively inferior and superior to the eye, symmetrically with respect to the optical axis, in the same vertical plane as each other.

Ocular biometry system 800 may also comprise a control system similar to that described with reference to FIG. 3 that operates in a similar manner with regard ocular biometry system 800 as is described for control system 208 in relation to system 200.

The projection of light beams 803 into the eye 801, the capture of images of the light beams when passing through the eye using cameras 807, and the calculation of optical path lengths and geometric path lengths to determine parameters of the eye is performed in a similar manner to that described in relation to ocular biometry system 200 above. Since two light beams 803 are incident on the eye, reflection points A, B, C and D are determined for each light beam, represented as A₁, A₂, B₁, B₂, etc in FIGS. 8 and 9. Assuming the light beams are projected substantially symmetrically into the eye and the lens is substantially symmetric in the plane of the light beams, retinal reflection points D₁ and D₂ collocate but are treated separately mathematically in the calculations to determine geometric path lengths.

The parameters of the eye determined by applying the above-described method to the image data captured from ocular biometry system 800 are parameters of the eye at the positions at which the light beams 803 pass through the eye.

Multiple Light Sources and Multiple Light Beam Incidence Positions

FIG. 10 is a side view schematic illustration of an ocular biometry system 900 according to another embodiment of the invention. Ocular biometry system 900 in FIG. 10 is similar to the system shown in FIG. 9 but with the orientation of reflectors 804 being able to be adjusted, as indicated by arrow 809. In this embodiment system 900 comprises first and second beam adjustment mechanisms configured to adjust the orientation of reflectors 804a and 804 respectively into a plurality of positions so that light beams 803 are incident on eye 801 in a plurality of positions. A control system controls the beam adjustment mechanism to adjust the orientations of reflectors 804 to achieve the desired range of light beam incident positions. In the embodiment shown the beam adjustment mechanisms are configured to rotate reflectors 804 around a horizontal axis in the direction of arrow 809 in order to adjust the direction of light beams 803 in the vertical plane. In other embodiments the beam adjustment mechanisms are configured to adjust reflectors 804 in other manners in order to adjust the direction of lights beams 803 in another direction, e.g. in the horizontal plane. More generally, the first and second beam adjustment mechanisms may be configured to adjust the position of reflectors 804 as well as, or instead of, their orientation, and/or the position/orientation of light sources 802.

In certain embodiments, the system 800 may comprise one or more lenses or other optical components configured to cause all light beams from the same light source 802 incident on eye 801 to travel in parallel. For example, the light beams may all be parallel to the optical axis of the eye. This may be achieved in one embodiment by locating a lens between reflector 804 and eye 801 with the point of reflection of the light beam from the reflector 804 being the focal point of the lens. In another embodiment two reflectors for each light beam may be used, with the focal point of the lens being located between the reflectors. In other embodiments, another afocal arrangement of optical components acting on incident light may be provided. As has been explained above, such arrangements may be advantageous to ensure the optical properties of the light beam entering the eye are the same as those of the light beam generated by the light source.

Eye parameters are determined for multiple light beam incidence positions using the system shown in FIG. 10 in a similar manner to that described previously. For example, the co-ordinates of reflection points A_{1 . . . n}, B_{1 . . . n}, C_{1 . . . n} and D_{1 . . . n} are determined for each of the pairs of light beams 803 in each of the plurality of incidence positions achieved by adjustment of reflectors 804, in the manner of a raster scan. This provides an array of co-ordinate points and eye parameters that enables a three-dimensional model of the eye to be constructed and displayed on a suitable display device. Alternatively or additionally, data indicative of the three-dimensional model of the eye is stored on a data storage device or communicated to another device, for example over a communications network. The three-dimensional model of the eye may be used to assess eye conditions such as refractive errors, e.g. myopia and astigmatism.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

1. An ocular biometry system comprising:
a light source configured to generate a light beam for incidence on an eye;
a beam adjustment mechanism configured to adjust the light beam to be incident on the eye in a plurality of incidence positions, wherein in at least one of the plurality of incidence positions the light beam is incident on the eye non-centrally;
first and second cameras configured to capture a plurality of images of the eye, each of the plurality of images being of the light beam passing through the eye when the light beam is in a different incidence position of the plurality of incidence positions; and
one or more processors configured to:
identify a plurality of features in each of the plurality of images, each of the plurality of features being representative of the light beam passing from one part of the eye to another part of the eye; and
determine, from the identified plurality of features in the plurality of images, one or more parameters of the eye.

2. An ocular biometry system as claimed in claim 1, wherein the parameters determined by the one or more processors are one or more of the parameters selected from the group consisting of: axial length; anterior chamber depth; posterior chamber depth; lens thickness; corneal radius/curvature; anterior lens radius/curvature; posterior lens radius/curvature; and retinal radius/curvature.

3. An ocular biometry system as claimed in claim 1, wherein the plurality of features identified in the captured images are representative of the light beam passing from and to one or more of the parts of the eye selected from the group consisting of: cornea; anterior chamber (aqueous humor); posterior chamber; lens; vitreous humor; and retina.

4. An ocular biometry system as claimed in claim 1, wherein the one or more processors identify the plurality of features in the captured images by identifying regions of relatively high intensity light in the captured images, the regions of relatively high intensity light corresponding to the plurality of features.

5. An ocular biometry system as claimed in claim 1, wherein the one or more processors determine an optical path length between two locations in the eye from positions of the features in the captured images, and calculate a geometric path length between the two locations in the eye from the optical path length.

6. (canceled)

7. An ocular biometry system as claimed in claim 1, wherein the beam adjustment mechanism comprises:
a reflector, the light beam being reflected by the reflector before entering the eye; and
a reflector adjustment mechanism configured to adjust the orientation and/or position of the reflector.

8. An ocular biometry system as claimed in claim 1, wherein the light source comprises one or more light sources configured to generate the light beam, wherein the light beam is a first light beam for incidence on the eye, and the one or more light sources are further configured to generate a second light beam for incidence on the eye, the first and second light beams being separated by a distance when incident on the eye, and further wherein the first and second cameras are configured to capture images of the eye when the first and second light beams pass through the eye.

9. (canceled)

10. (canceled)

11. An ocular biometry system as claimed in claim 8, wherein the one or more light sources are configured such that the first and second light beams are incident on the eye symmetrically with respect to an axis of the eye.

12. An ocular biometry system as claimed in claim 8, wherein the beam adjustment mechanism is configured to adjust the first and second light beams to be incident on the eye in a plurality of incidence positions, wherein each of the plurality of images is of the first and/or second light beams passing through the eye when the first and second light beams are in different incidence positions of the plurality of incidence positions.

13. An ocular biometry system as claimed in claim 12, wherein the beam adjustment mechanism comprises a first beam adjustment mechanism configured to adjust the first light beam to be incident on the eye in a plurality of incidence positions and a second beam adjustment mechanism configured to adjust the second light beam to be incident on the eye in a plurality of incidence positions.

14. An ocular biometry system as claimed in claim 8, wherein the ocular biometry system comprises third and fourth cameras configured to capture images of the eye when the first and second light beams pass through the eye.

15. An ocular biometry system as claimed in claim 14, wherein the first and third cameras are positioned symmetrically relative to the eye and are configured to capture images of a first set of parts of the eye, and the second and fourth cameras are positioned symmetrically relative to the eye and are configured to capture images of a second set of parts of the eye.

16.-20. (canceled)

21. A processor-implemented method of measuring a parameter of an eye, the method comprising:
receiving a plurality of images of the eye, each of the plurality of images being of a light beam passing through the eye when the light beam is in one of a plurality of incidence positions, wherein in at least one of the plurality of incidence positions the light beam is incident on the eye non-centrally;
identifying a plurality of features in each of the plurality of images, each of the plurality of features being representative of the light beam passing from one part of the eye to another part of the eye; and
determining the parameter from the identified plurality of features in the plurality of images.

22. A processor-implemented method as claimed in claim 21,
wherein the method comprises determining one or more parameters of the eye, the parameters being one or more of the parameters selected from the group consisting of: axial length; anterior chamber depth; posterior chamber depth; lens thickness; corneal radius/curvature; anterior lens radius/curvature; posterior lens radius/curvature; and retinal radius/curvature.

23. A processor-implemented method as claimed in claim 21, wherein the plurality of features identified in the images are representative of the light beam passing from and to one or more of the parts of the eye selected from the group consisting of: cornea; anterior chamber (aqueous humor); posterior chamber; lens; vitreous humor; and retina.

24. A processor-implemented method as claimed in claim 21, wherein the method comprises identifying the plurality of features in the images by identifying regions of relatively high intensity light in the captured images, the regions of relatively high intensity light corresponding to the plurality of features.

25. A processor-implemented method as claimed in claim 21, wherein the method comprises determining an optical path length between two locations in the eye from positions of the features in the images, and calculating a geometric path length between the two locations in the eye from the optical path length.

26. A processor-implemented method as claimed in claim 21, wherein the method comprises:
controlling a beam adjustment mechanism to adjust the light beam to be incident on the eye in the plurality of incidence positions.

27. A processor-implemented method as claimed in claim 21, wherein the method comprises:
controlling a first beam adjustment mechanism to adjust a first light beam to be incident on the eye in a plurality of incidence positions; and
controlling a second beam adjustment mechanism to adjust a second light beam to be incident on the eye in a plurality of incidence positions.

28. (canceled)

29. (canceled)

30. A method of measuring a parameter of an eye, the method comprising:
shining a light beam into the eye;
adjusting the light beam to be incident on the eye in a plurality of incidence positions, wherein in at least one of the plurality of incidence positions the light beam is incident on the eye non-centrally;
capturing a plurality of images of the eye, each of the plurality of images being of the light beam passing through the eye when the light beam is in a different incidence position of the plurality of incidence positions;
identifying a plurality of features in each of the plurality of images, each of the plurality of features being representative of the light beam passing from one part of the eye to another part of the eye; and
determining the parameter from the identified plurality of features in the plurality of images.

31.-44. (canceled)
